(19) United States
(12) Patent Application Publication
(10) Pub. No.: US 2022/0168262 A1
(43) Pub. Date: Jun. 2, 2022

(54) AMPHIPHILIC PLATINUM (IV) PRODRUG AND CANCER TREATMENT PROCESS

(71) Applicant: Kent State University, Kent, OH (US)

(72) Inventors: Yaorong ZHENG, Kent, OH (US); Zhizhou YUE, Newtown, PA (US)

(73) Assignee: Kent State University, Kent, OH (US)

(21) Appl. No.: 17/593,472

(22) PCT Filed: Mar. 17, 2020
(86) PCT No.: PCT/US2020/023085

§ 371 Date: Sep. 20, 2021

Related U.S. Application Data
(60) Provisional application No. 62/819,752, filed on Mar. 18, 2019.

(51) Int. Cl. A61K 31/282; A61K 35/15; A61K 9/00; A61K 41/00

(52) U.S. Cl. CPC A61K 31/282; A61K 41/0042; A61K 9/0019; A61K 35/15

(57) ABSTRACT

A fatty acid-like platinum (IV) prodrug is useful for treating cancer (e.g., ovarian cancer). The fatty acid-like platinum (IV) prodrug may be used in combination with a M1 macrophage.

This application which claims the benefit of U.S. Provisional Application Ser. No. 62/819,752, filed Mar. 18, 2019 and titled “FATTY ACID-LIKE PLATINUM (iv) PRODRUG AND CANCER TREATMENT PROCESS,” the entirety of which is hereby expressly incorporated by reference herein.

### BACKGROUND

The present disclosure relates to fatty acid-like platinum (IV) prodrugs. The prodrugs may be useful for treating cancer (e.g., chemoresistant ovarian cancer).

Ovarian cancer is the most lethal gynecological cancer. Twenty-thousand women will be diagnosed each year. Diagnosis frequently occurs after the cancer has already metastasized. The five-year survival rate for ovarian cancer (e.g., ˜40-50%) has not significantly changed for decades.

FDA-approved treatments include cisplatin:

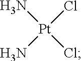

and carboplatin:

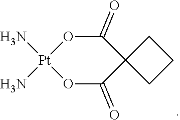

One weakness of current chemotherapeutics is that transport across the plasma membrane is difficult.

Some known treatments is that they kill tumor stem cells but not cancer stem cells. Unfortunately, this can lead to the tumor shrinking but eventually growing back.

Chemoresistance is a challenge in the treatment of ovarian cancer. Currently, there is no promising way to treat chemoresistant ovarian cancer. Once chemoresistance develops, patients are deemed by many to be incurable due to the lack of treatments.

It would be desirable to develop new compositions and methods for treating cancer, particularly ovarian cancer.

### BRIEF DESCRIPTION

The present disclosure relates to a fatty acid-like platinum (IV) prodrug, a composition containing the prodrug, a method for manufacturing the prodrug, and a method for treating a patient using the prodrug.

Disclosed, in some embodiments, is a fatty acid-like Pt(IV) prodrug of Formula I

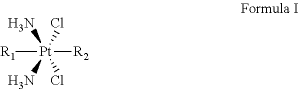

wherein R₁ and R₂ are axial ligands.

R₁ and R₂ may be the same or different.

A method for treating cancer may include administering the prodrug to a patient diagnosed with cancer. The cancer may be ovarian cancer. The prodrug may be administered to the patient via injection into a vein.

Disclosed, in other embodiments, is a prodrug of Formula II

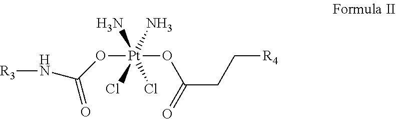

wherein R₃ and R₄ are independently selected from the group consisting of alkyl, alkenyl, —COOH, —NHR₅ wherein R₅ is alkyl,

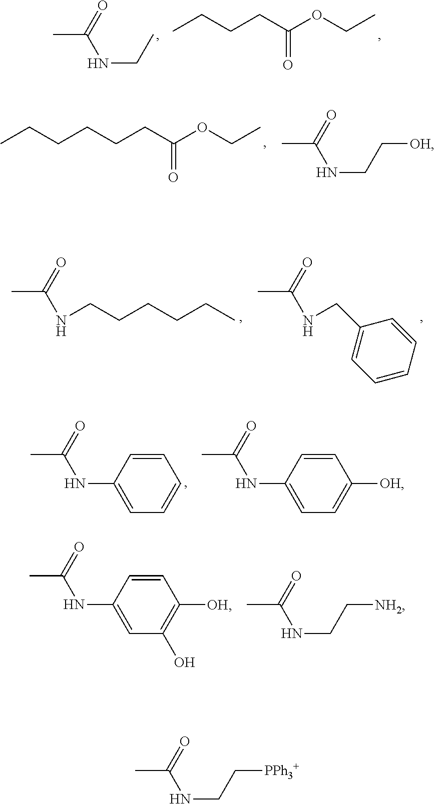

wherein PPh₃ is triphenylphosphine, and

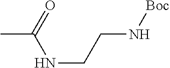

wherein Boc is tert-butyloxycarbonyl.

R₃ and R₄ may be the same or different.

Disclosed, in further embodiments, is a fatty acid-like platinum (IV) prodrug selected from the group consisting of:

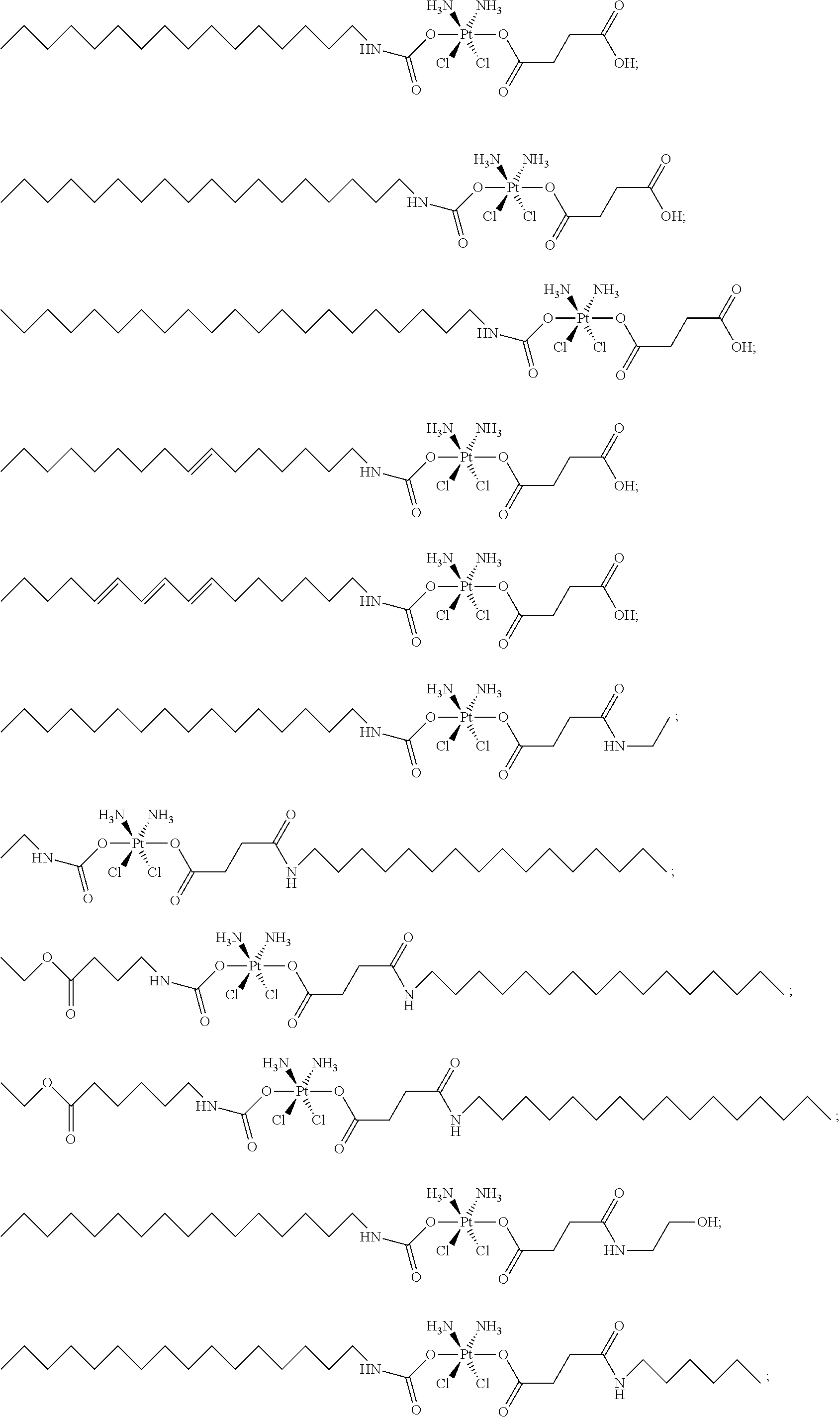

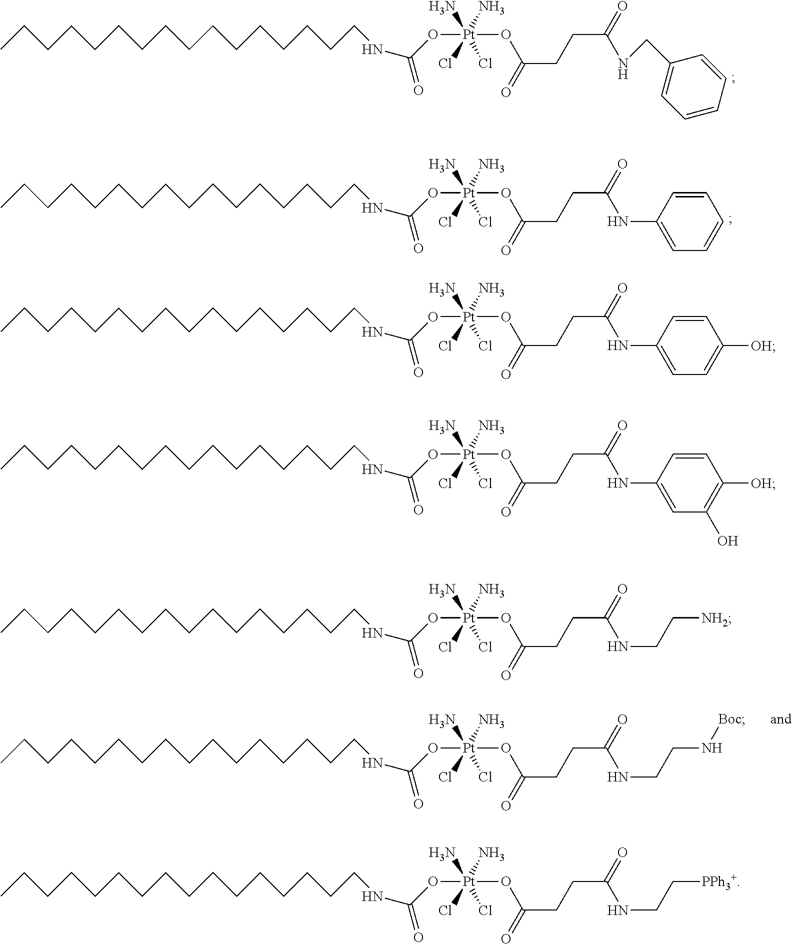

The treatment methods of the present disclosure may further include administering a M1 macrophage to the patient or polarizing a macrophage to be a M1 macrophage in vitro.

The prodrug and the macrophage may be administered together or separately.

In some embodiments, the prodrug is conjugated with a TLR agonist.

These and other non-limiting characteristics are more particularly described below and in the appended materials.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 illustrates the CD36-dependent cellular uptake of amphiphilic Pt(IV) prodrugs.

FIG. 2 illustrates the photoactivation of the amphiphilic prodrugs.

FIG. 3 illustrates cytotoxicity profiles of the amphiphilic prodrugs.

### DETAILED DESCRIPTION

The present disclosure may be understood more readily by reference to the following detailed description of desired embodiments included therein and the appended slides. In the following specification and the claims which follow, reference will be made to a number of terms which shall be defined to have the following meanings.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent can be used in practice or testing of the present disclosure. All publications, patent applications, patents, and other references mentioned herein are incorporated by reference in their entirety. The materials, methods, and articles disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases that require the presence of the named ingredients/steps and permit the presence of other ingredients/steps. However, such description should be construed as also describing compositions, mixtures, or processes as “consisting of” and “consisting essentially of” the enumerated ingredients/steps, which allows the presence of only the named ingredients/steps, along with any impurities that might result therefrom, and excludes other ingredients/steps.

Unless indicated to the contrary, the numerical values in the specification should be understood to include numerical values which are the same when reduced to the same number of significant figures and numerical values which differ from the stated value by less than the experimental error of the conventional measurement technique of the type used to determine the particular value.

All ranges disclosed herein are inclusive of the recited endpoint and independently combinable (for example, the range of “from 2 to 10” is inclusive of the endpoints, 2 and 10, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Ovarian tumors preferentially metastasize to omentum. Adipocytes promote ovarian cancer metastasis and provide energy for rapid tumor growth. Adipocytes upregulate CD36 expression in SKOV-3 cells. SKO-3 is an ovarian cancer cell line. Upregulated CD36 facilitates fatty acid uptake in SKOV-3.

Platinum (IV) prodrugs may have an inert Pt(IV) core obtained from the oxidation of Pt(II) drugs. Additional ligands may be useful to tune the chemical/physical properties. Intracellular reduction may be required to allow Pt(IV) prodrugs to release their active Pt contents.

Non-limiting examples of advantages include facile synthesis (no column chromatography is needed for most steps), decent yields (e.g., 70-90% in each step), and stable products under various conditions (e.g., the end products are stable in 20% TFA/DCM for hours).

In some embodiments, the fatty acid-like Pt(IV) prodrug is of Formula I

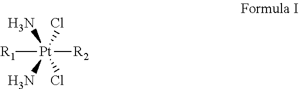

wherein R₁ and R₂ are axial ligands.

In some embodiments, the fatty acid-like Pt(IV) prodrug is selected from at least one of the following:

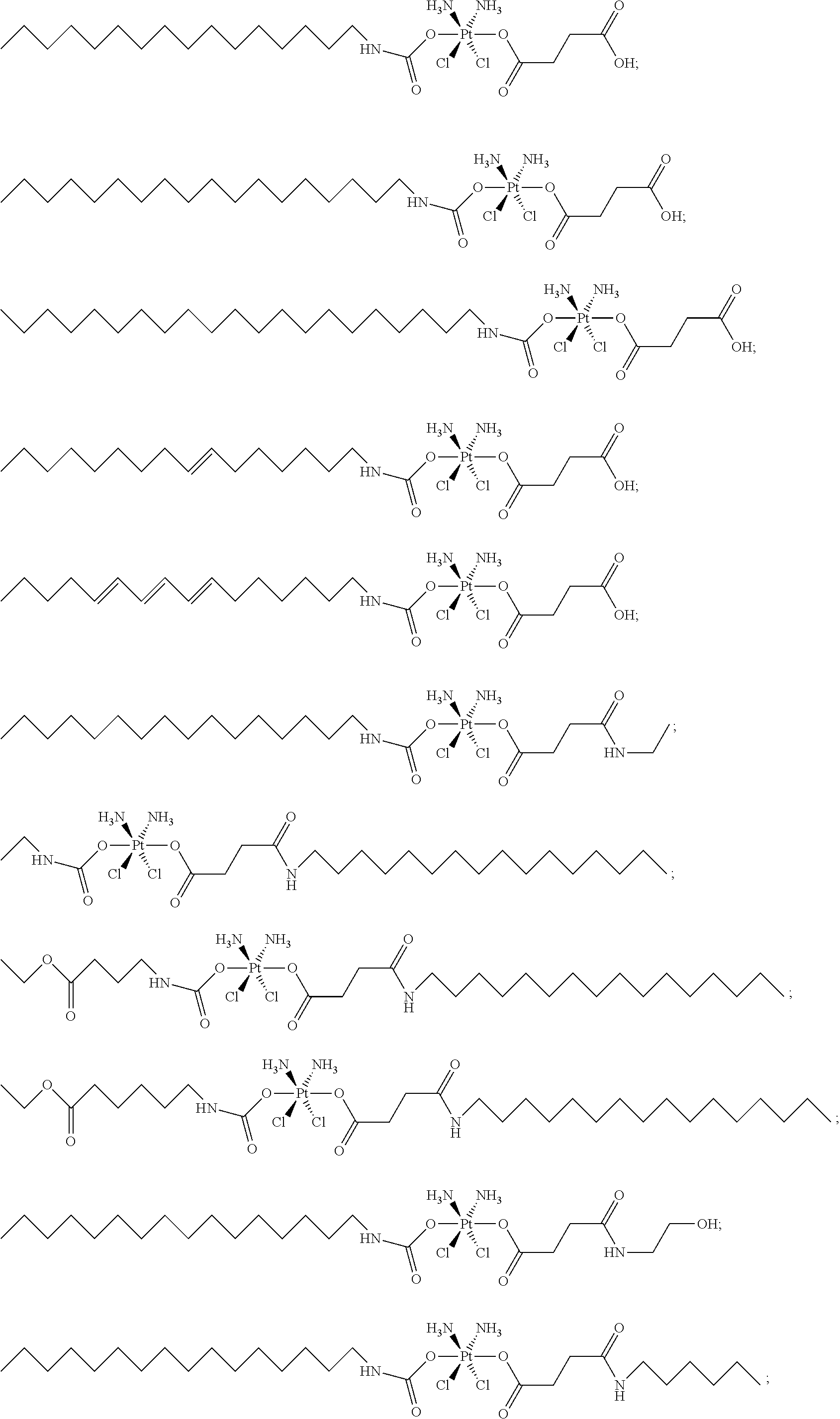

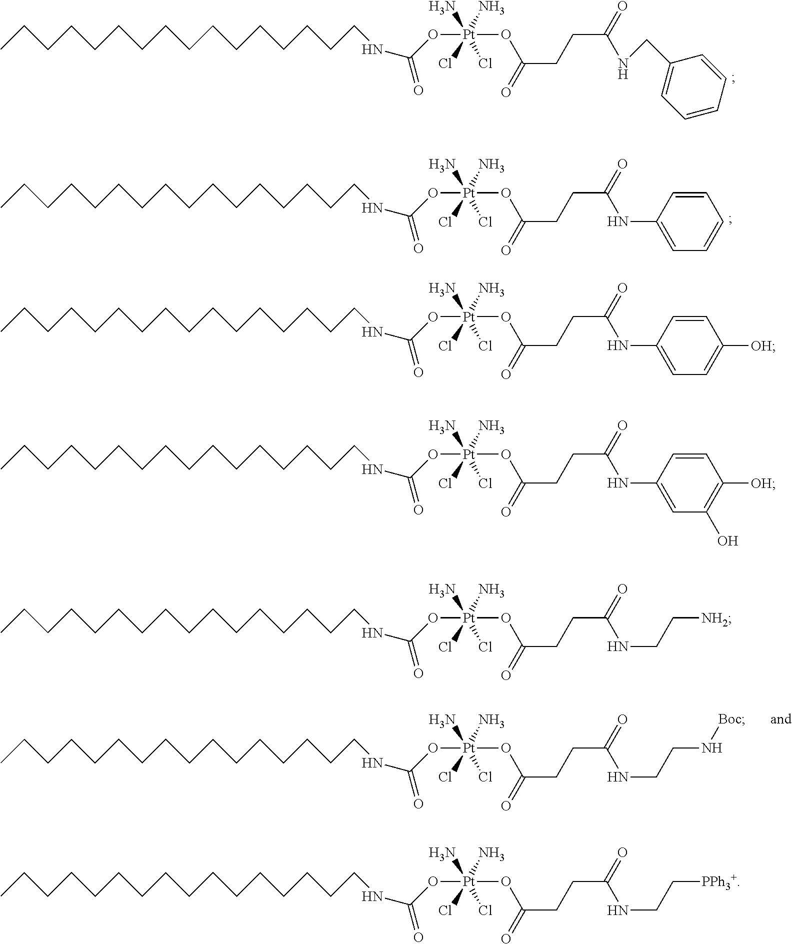

In some embodiments, the fatty acid-like Pt(IV) prodrug is of general Formula II wherein R₃ and R₄ have the meanings discussed below.

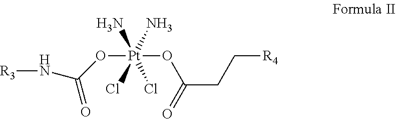

R₃ and R₄ may be independently selected from:

alkyl (e.g., C₁₀-C₂₂ alkyl, including C₁₃-C₁₉ alkyl, and C₁₆ alkyl), alkenyl, —COOH, —NHR₅ wherein R₅ is alkyl,

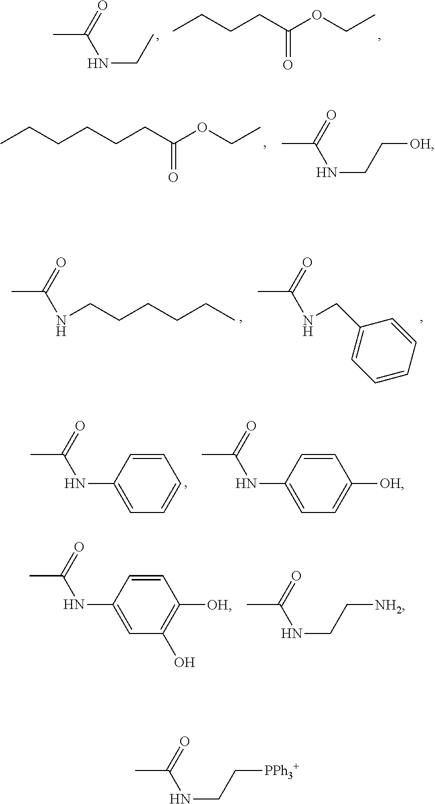

wherein PPh₃ is triphenylphosphine, and

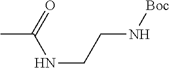

wherein Boc is tert-butyloxycarbonyl.

In some embodiments, R₃ is alkyl and R₄ is not alkyl. In these embodiments, R₂ may be selected from —COOH, —NHR₅ wherein R₅ is alkyl,

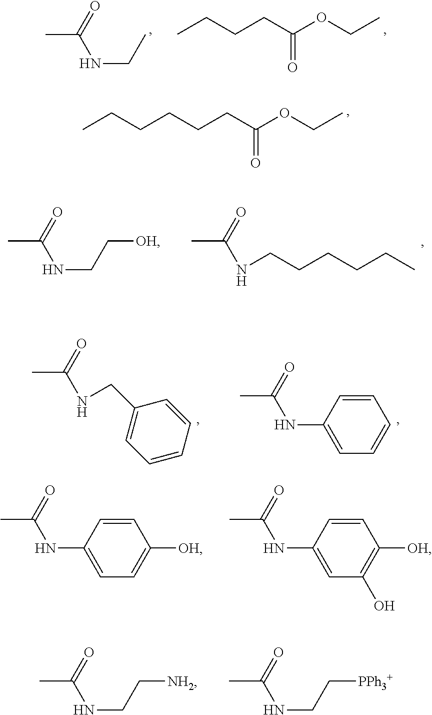

wherein PPh₃ is triphenylphosphine, and

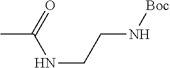

wherein Boc is tert-butyloxycarbonyl.

In other embodiments, R₄ is alkyl and R₃ is not alkyl. In these embodiments, R₁ may be selected from —COOH, —NHR₅ wherein R₅ is alkyl,

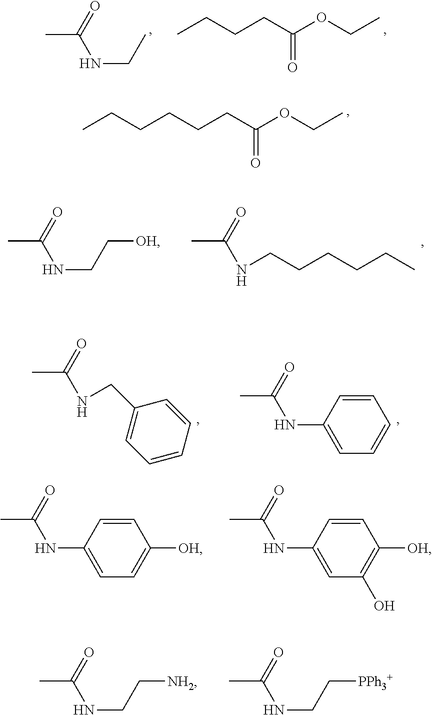

wherein PPh₃ is triphenylphosphine, and

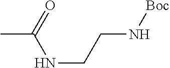

wherein Boc is tert-butyloxycarbonyl.

The prodrugs may be reduced by various biological reductants (e.g., glutathione, ascorbate), the t_{1/2} can be hours, and the reduction products can include cisplatin, amine, carboxylate, and CO₂. Cisplatin is a FDA-approved platinum chemotherapeutic. Carboplatin is another FDA-approved chemotherapeutic.

One weakness of current chemotherapeutics is that transport across the plasma membrane is difficult.

Advantageously, the Pt(IV) prodrugs of the present disclosure may exhibit higher SKOV-3 cell uptake compared to cisplatin. Uptake may be further enhanced by exposing the SKOV-3 cells to conditioned medium. Although the treatment methods of the present disclosure are described in reference to SKOV-3 cells, it should be understood that other types of cells are also contemplated.

In some embodiments, the Pt(IV) prodrug may have a 50% inhibitory concentration (IC₅₀) for SKOV-3 cells of less than 40 μM, including less than 35 μM, and less than 30 μM.

In some embodiments, the Pt(IV) prodrug combined with conditioned medium treatment may have an IC₅₀ for SKOV-3 cells of less than 30, including less than 25 μM, less than 20 μM, less than 15 μM, and less than 12 μM.

In some embodiments, the prodrugs are hydrophobic, cationic Pt(IV) prodrugs designed to target mitochondria.

Unfortunately, when a drug kills tumor cells but not cancer stem cells, the tumor may shrink but eventually grow back. Advantageously, the Pt(IV) prodrugs of the present disclosure may eliminate cancel stem cells.

In some embodiments, nanoparticles of the Pt(IV) prodrugs may be used in combination with macrophages (e.g., tumor-associated macrophages). The macrophages may serve as “drug depots” by accumulating the nanoparticles and releasing the Pt payload to surrounding tumor cells.

The macrophages may be M1 (classically activated macrophages) or M2 (alternatively activated macrophages). In particular embodiments, the macrophages are M1 macrophages. M1 macrophages may exhibit anti-tumor effects against ovarian cancer cells.

In some embodiments, the M1 macrophages are produced by treating macrophages with M1 polarizing agents.

Without wishing to be bound by theory, it appears that Pt(IV) prodrugs such as C16Pt may enhance the anti-tumor activities of M1 macrophages by increasing mitochondrial production of reactive oxygen species, thereby leading to elevated nitric oxide and enhanced anti-tumor activities.

C16Pt appears to respond synergistically with a series of TLR agonists against ovarian cancer via polarizing macrophages to M1 phenotype. The therapeutic effect of the combination of fatty acid-like Pt(IV) prodrugs and M1 polarizing agents may be superior to that of cisplatin.

In vitro, C16Pt can spare M1 macrophages while killing chemoresistant ovarian cancer cells. M1 macrophages exhibit anti-tumor effects against ovarian cancer cells. C16Pt enhances the anti-tumor activities of M1 macrophages. C16Pt synergizes with TLR agonists against ovarian cancer via polarizing macrophages.

In some embodiments, one of the axial ligands of the Pt(IV) prodrug includes a long (e.g., C16) alkyl chain and the other axial ligand is conjugated with a TLR agonist.

The TLR agonist may be a TLR 1 agonist, a TLR2 agonist, a TLR3 agonist, a TLR4 agonist, a TLR5 agonist, a TLR6 agonist, a TLR7 agonist, a TLR8 agonist, and/or a TLR9 agonist. In some embodiments, the TLR agonist is known to activate TLR7 and/or TLR8.

Non-limiting examples of TLR agonists include:

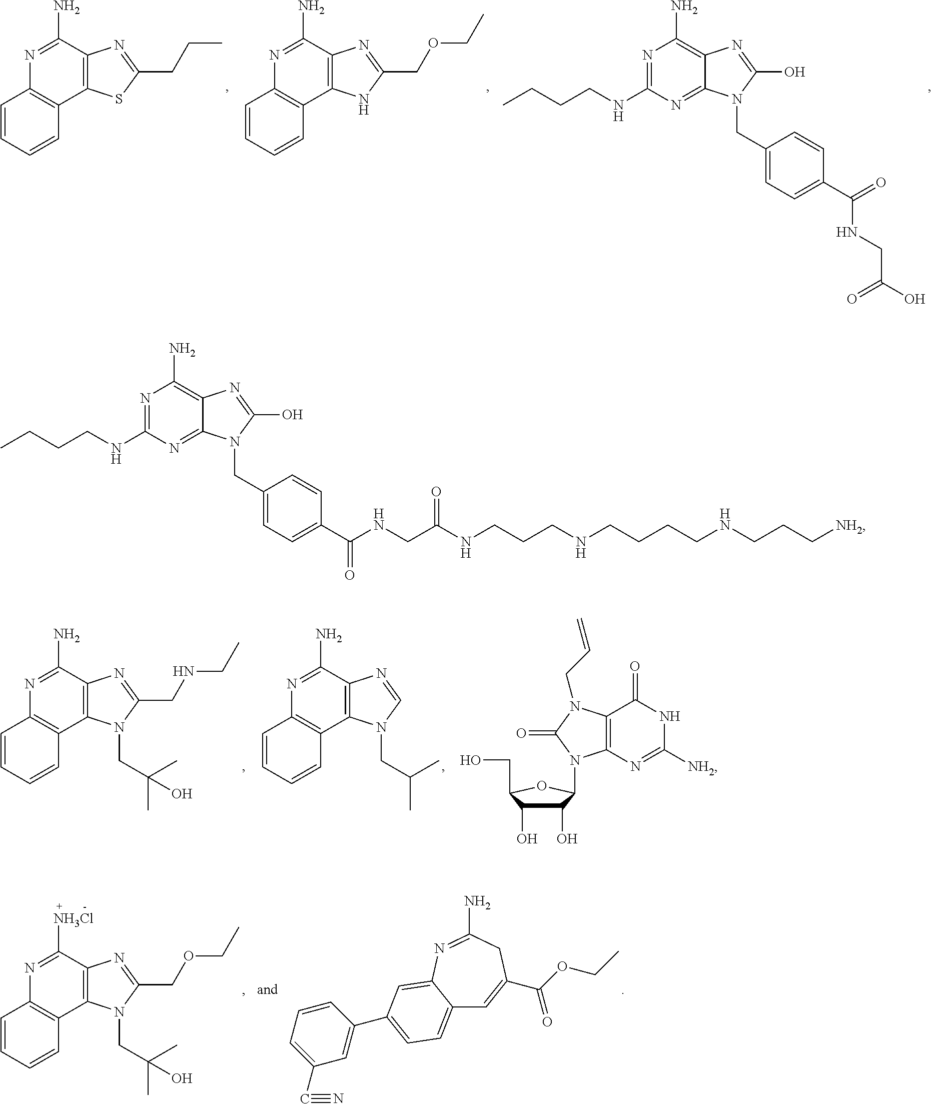

The following examples are provided to illustrate the compositions and methods of the present disclosure. The examples are merely illustrative and are not intended to limit the disclosure to the materials, conditions, or process parameters set forth therein.

### EXAMPLES

Amphiphilic Pt(IV) prodrugs were studied. The prodrugs may be visible light activatable and may harness CD36 for ovarian cancer therapy.

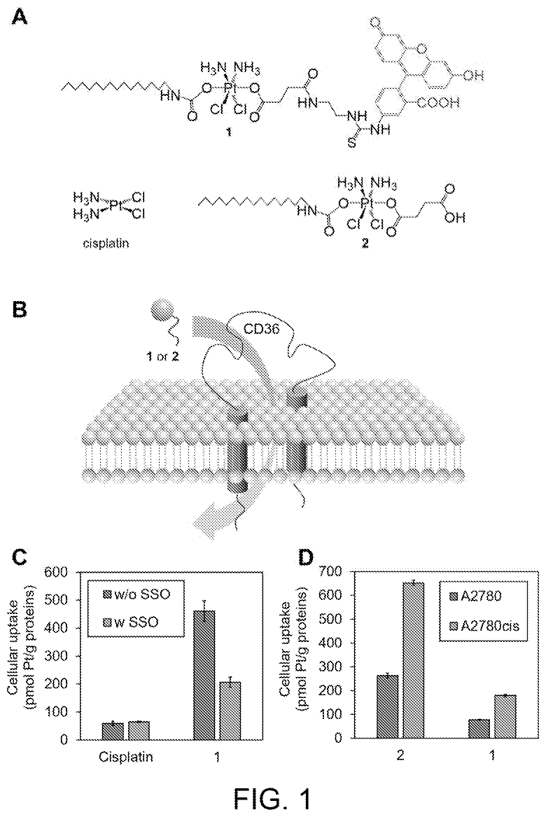

FIG. 1 illustrates the CD36-dependent cellular uptake of amphiphilic Pt(IV) prodrugs. FIG. 1A shows the chemical structures of the compounds involved in the study. FIG. 1B is a graphical representation of the CD36-dependent cell entry of the compounds. FIG. 1C illustrates the GFAAS analysis of cellular uptake of cisplatin and compound 1 of FIG. 1A without sulfosuccinimidyl oleate (SSO, left) and with SSO (right). FIG. 1D illustrates the GFAAS analysis of cellular uptake of compounds 1 and 2 of FIG. 1A in A2780 and A2780cis ovarian cancer cells ([Pt]=10 μM, 24 h, 37°, 5% CO₂).

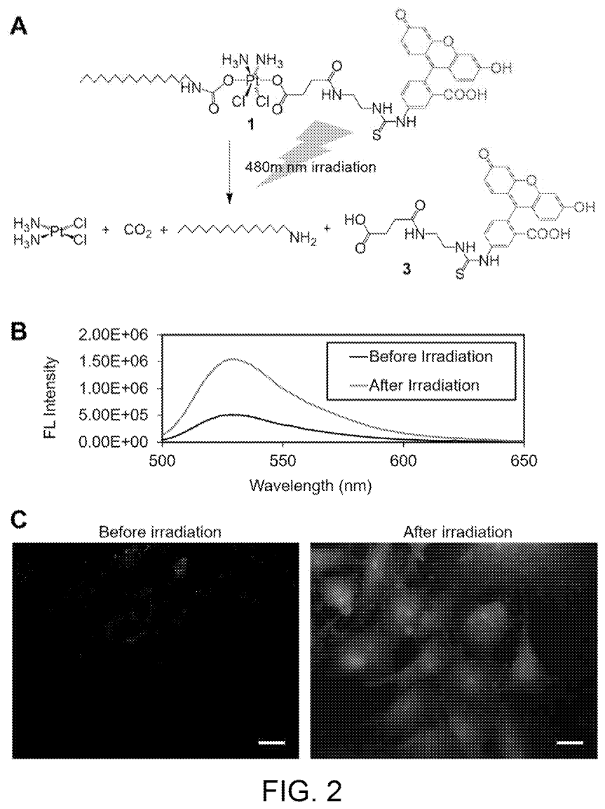

FIG. 2 illustrates the photoactivation of the amphiphilic prodrugs. FIG. 2A is a graphical representation of the photoactivation process. FIG. 2B is a graph showing the fluorescence spectra of compound 1 recorded before (lower curve) and after (upper curve) 20-min irradiation by 480 nm LED. FIG. 2C are fluorescence images of A549 cells treated with compound 1 ([Pt]=10 μM, 24 h, 37° C., 5% CO₂).

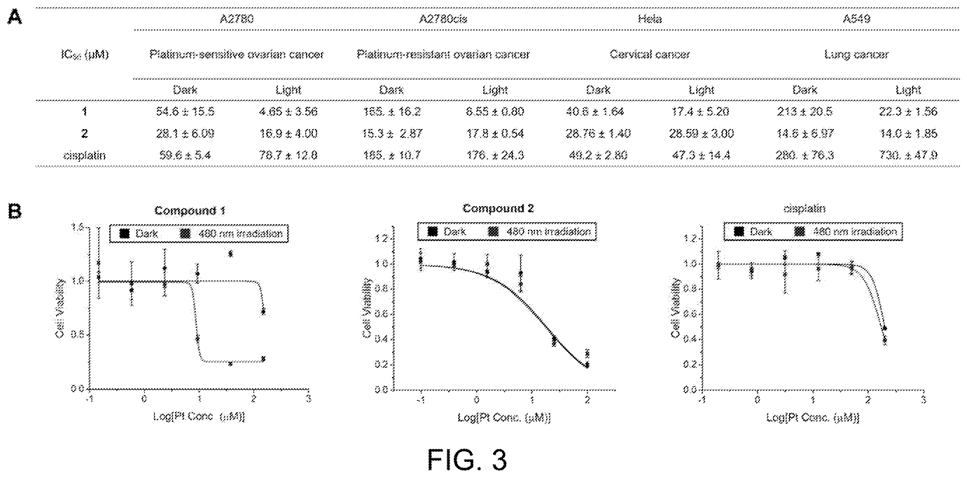

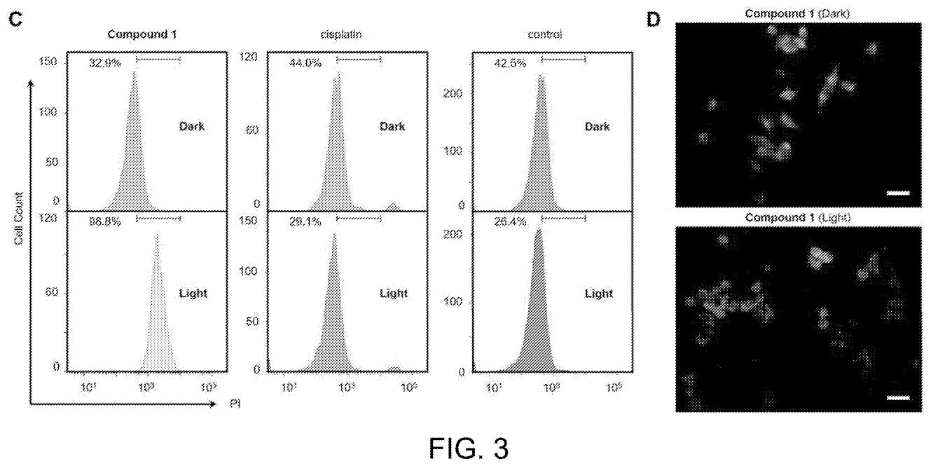

FIG. 3 illustrates cytotoxicity profiles of the amphiphilic prodrugs. FIG. 3A is a table of IC₅₀ of cisplatin and compound 1 or compound 2 against A2780 (cisplatin-sensitive), A2780cis (cisplatin-resistant) ovarian cancer cells, Hela cervical cancer cells, and A549 lung cancer cells with 24-h incubation. FIG. 3B are graphs showing the representative killing curves of MTT assays with and without irradiation. FIG. 3C illustrates the flow cytometric analysis of propidium iodide in A2780cis cells treated with compound 1 or cisplatin ([Pt]=10 μM, 24 h, 37° C., 5% CO₂). FIG. 3D are fluorescence images of Live/Dead cell assays of A2789cis treated with compound 1 or cisplatin ([Pt]=10 μM, 24 h, 37° C., 5% CO₂).

It will be appreciated that variants of the above-disclosed and other features and functions, or alternatives thereof, may be combined into many other different systems or applications. Various presently unforeseen or unanticipated alternatives, modifications, variations or improvements therein may be subsequently made by those skilled in the art which are also intended to be encompassed by the following claims.

1. A fatty acid-like platinum (IV) prodrug for treating cancer.

2. The fatty acid-like platinum (IV) prodrug of claim 1, wherein the prodrug is of Formula I 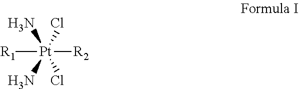
wherein R₁ and R₂ are axial ligands.

3. The prodrug of claim 2, wherein R₁ and R₂ are different.

4. A method for treating cancer, the method comprising:
administering the prodrug of claim 2 to a patient diagnosed with cancer.

5. The method of claim 4, wherein the cancer is ovarian cancer.

6. The method of claim 4, wherein the prodrug is administered to the patient via injection into a vein.

7. A prodrug of Formula II 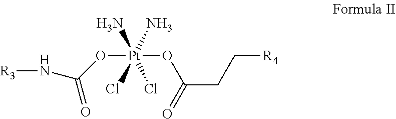
wherein R₃ and R₄ are independently selected from the group consisting of alkyl, alkenyl, —COOH, —NHR₅ wherein R₅ is alkyl, 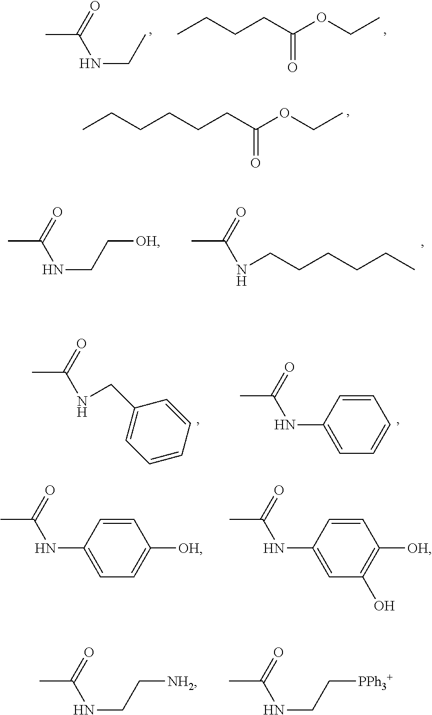
wherein PPh₃ is triphenylphosphine, and 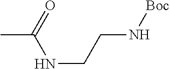
wherein Boc is tert-butyloxycarbonyl.

8. The prodrug of claim 7, wherein R₃ and R₄ are different.

9. A method for treating cancer, the method comprising:
administering the prodrug of claim 7 to a patient diagnosed with cancer.

10. The method of claim 9, wherein the cancer is ovarian cancer.

11. The method of claim 9, wherein the prodrug is administered to the patient via injection into a vein.

12. A fatty acid-like platinum (IV) prodrug selected from the group consisting of:

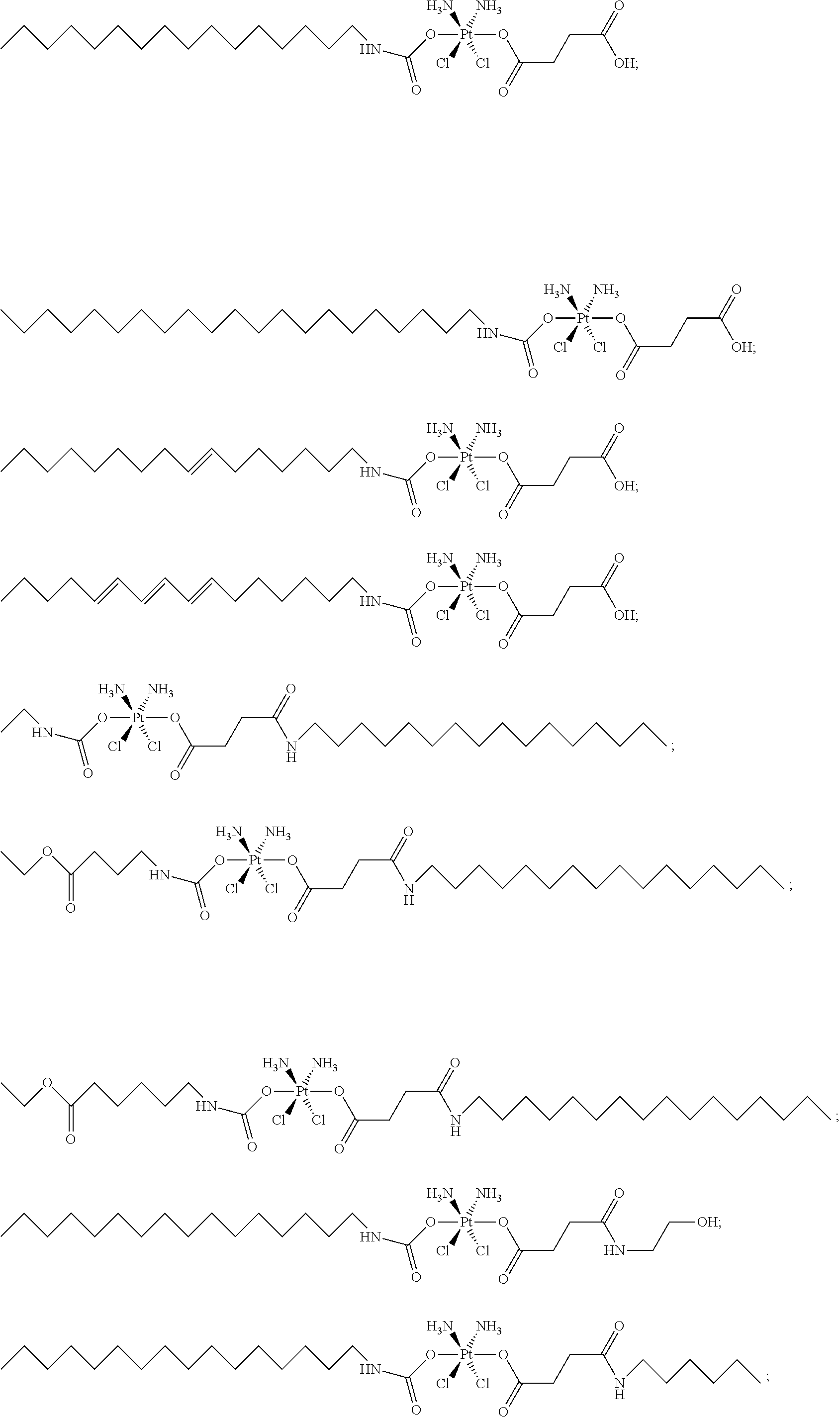

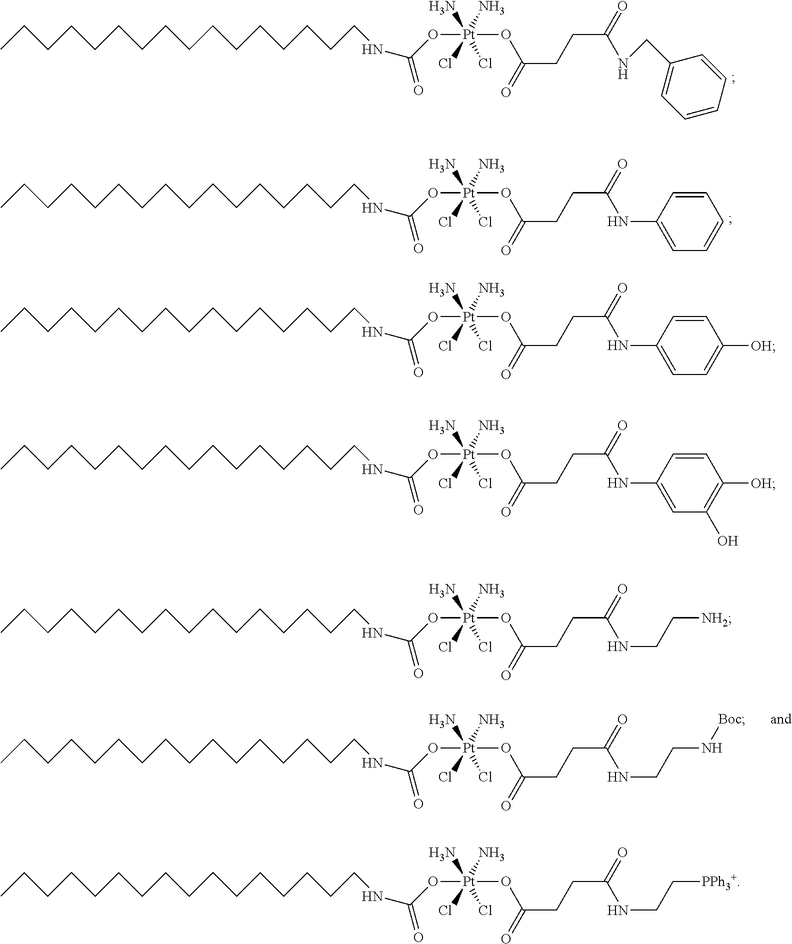

13. A method for treating cancer, the method comprising:
administering the prodrug of claim 12 to a patient diagnosed with cancer.

14. The method of claim 13, wherein the cancer is ovarian cancer.

15. The method of claim 13, wherein the prodrug is administered to the patient via injection into a vein.

16. The method of claim 13, further comprising:
administering a M1 macrophage to the patient.

17. The method of claim 16, wherein the prodrug and the macrophage are administered together.

18. The method of claim 16, wherein the prodrug and the macrophage are administered separately.

19. The method of claim 13, wherein the prodrug is conjugated with a TLR agonist.

20. A cancer treatment method comprising:
intravenously administering the fatty acid-like platinum (IV) prodrug of claim 1 to a cancer patient.
